(19) United States
(12) Patent Application Publication
(10) Pub. No.: US 2022/0306732 A1
(43) Pub. Date: Sep. 29, 2022

(54) MODIFIED DOSING OF VEGF INHIBITORS FOR OPHTHALMIC USE

(71) Applicant: Lupin Limited, Mumbai (IN)

(72) Inventors: Dhananjay Sadashiv BAKHLE, Pune (IN); Chirag Anilkumar SHAH, Pune (IN); Preetam Nivrutti CHAVAN, Pune (IN)

(73) Assignee: Lupin Limited, Mumbai (IN)

(21) Appl. No.: 17/638,966

(22) PCT Filed: Sep. 10, 2020
(86) PCT No.: PCT/IB2020/058409

§ 371 Date: Feb. 28, 2022

(30) Foreign Application Priority Data
Sep. 12, 2019 (IN) 201921036781

(51) Int. Cl. C07K 16/22; A61P 27/02; A61K 9/00

(52) U.S. Cl. CPC C07K 16/22; A61P 27/02; A61K 9/0048; A61K 2039/505

(57) ABSTRACT

The present invention provides a method of treating ocular disorders by administering a suitable amount of VEGF inhibitors in a patient in need thereof, wherein the suitable amount of VEGF inhibitor is determined with respect to the body weight of the patient. Further, present invention also provides a method of treating ocular disorders by administering a personalized dose of a suitable amount of VEGF inhibitors intravitreally, wherein the dose is decided based on the body weight of the said patient at any given point in time.

### FIELD OF THE INVENTION

The invention provides modified dosing of VEGF inhibitors for ophthalmic use. It also relates to methods of treating a VEGF associated ophthalmic disorders.

### BACKGROUND OF THE INVENTION

Increased levels of intra-vitreal VEGF has been associated with various angiogenic eye disorders, such as age-related macular degeneration (AMD) including both wet AMD, macular edema following retinal vein occlusion (RVO), diabetic macular edema (DME), diabetic retinopathy (DR) without or with DME and myopic choroidal neovascularization (mCNV).

VEGF promotes angiogenesis (new vessel formation) and is known to be a potent mediator of vascular permeability. VEGF is known to inhibit endothelial cells apoptosis, leading to the generation of immature vascular structures. These immature vascular structures are fragile, and hence easy to rupture causing bleedings, and favor retinal detachment and consequential blindness (Arch Med Sci. 2016 Oct. 1; 12(5)). Among various angiogenic eye disorders, age-related macular degeneration (AMD) and diabetic macular edema (DME) are amongst most prevalent. There are other prevalent eye disorders such as retinal vein occlusions and corneal neovascularization.

Age-related macular degeneration (AMD) is a common eye condition and a leading cause of vision loss among people age 50 and older. It causes damage to the macula, a small area in center of the retina and the part of the eye needed for sharp, central vision, which lets us see objects that are straight ahead.

Diabetic macular edema (DME), a manifestation of diabetic retinopathy that produces loss of central vision, is on the rise due to an increasing global burden of diabetes. Pathophysiology of DME relates to an abnormal leakage of fluid and macromolecules from retinal capillaries into the extravascular space. It further relates to the abnormality of the retinal pigment epithelium wherein the barrier to fluid flow from the choriocapillaries to the retina and also active pumping of fluid out of the retina gets affected.

Anti-vascular endothelial growth factor (anti-VEGF) agents have emerged as one of the key therapeutic drug classes for treating neovascular diseases of the eye. Pegaptanib sodium (Macugen) was the first Food and Drug Administration (FDA) approved anti-VEGF treatment for wet AMD. Ranibizumab (Lucentis) received FDA approval 2 years later in 2006. Minimally Classic/Occult Trial of the Anti-VEGF Antibody Ranibizumab in the Treatment of Neovascular AMD (MARINA) [N. Engl. J. Med. 355, 2006] and Anti-VEGF Antibody for the Treatment of Predominantly Classic CNV in AMD (ANCHOR) studies [Ophthalmology 116, 2009] established ranibizumab as the superior treatment for wet AMD, compared with any prior FDA approved treatments. Aflibercept (EYLEA), yet another anti-VEGF agent was approved by FDA in November 2011 indicated for the treatment for wet neovascular (Wet) age-related macular degeneration, macular edema following retinal vein occlusion (RVO), diabetic macular edema (DME) and diabetic retinopathy (DR). Brolucizumab (BEVOU) is one more VEGF inhibitor approved by FDA for the treatment of neovascular (wet) age-related macular degeneration.

Bevacizumab is a monoclonal antibody directed against VEGF which was developed for treatment of various cancers to inhibit angiogenesis. Since wet-AMD also show pathogenesis of angiogenesis due to presence of VEGF, use of bevacizumab in treating it was considered. On these lines, various academic institutions have conducted head-to-head clinical trials, comparing clinical efficacy of Bevacizumab with Ranibizumab in treating wet AMD. In this context, one of the most notable trials has been Comparison of Age-Related Macular Degeneration Treatment Trial (CATT Study). The study was conducted in 1200 AMD patients, and assessed the comparative efficacy of Ranibizumab (Lucentis) and Bevacizumab (Avastin) in treating AMD. The results of the study, published in NEJM, demonstrated non-inferiority of Bevacizumab to Ranibizumab in treatment of wet AMD. However, the CATT study utilized 1.25 mg bevacizumab during its study compared to 0.5 mg of Ranibizumab.

In another consideration, intra-vitreal use of Bevacizumab has been associated with two types of adverse effects; ocular (local side effect) and non-ocular (systemic side-effects due to leaking of Bevacizumab into circulation). The local side-effects include vitreous detachment, inflammation, hemorrhage and infection of eye (Shima et al, Acta Ophthalmologica, 86, (2008) while the most common systemic adverse events include hypertension, gastrointestinal symptoms (hemorrhage, nausea and vomiting), and upper respiratory infections (pneumonia) etc. Other associated adverse reactions incidence (>10%) are epistaxis, headache, hypertension, rhinitis, proteinuria, taste alternation, dry skin, rectal hemorrhage, lacrimation disorder, back pain and exfoliative dermatitis (Shima et al, Acta Ophthalmologica, 86, (2008).

The VEGF levels differ according to weight, being low for low weight and high for high weight patient. Conventional use of 1.25 mg Bevacizumab for treatment of low weight AMD patients would therefore lead to higher incidence of systemic adverse events due to leakage from eye cavity in the systemic circulation. These ocular adverse effects may be due to presence of excess Bevacizumab intravitreally, and consequential leakage from eye cavity into systemic circulation, contributing the systemic adverse effects.

Therefore, approaches to optimal dosing of VEGF inhibitors, especially Bevacizumab, may alleviate at least one of these disadvantages associated with the use of Bevacizumab.

In an approach to optimize the dose of bevacizumab, consideration of BMI/body weight of patient was taken into account. This was based on a fact shown in a clinical study conducted by Zhang et al (Ophthalmol. Vis Sci., March 2016; 57(3): 1276-83) which demonstrated that a mild positive correlation exists between body mass index (BMI) and circulating VEGF levels leading to an increased relative risk of AMD.

In another study, Loebig et al (PLoS One, September 2010, Vol 5(9): 1-5) demonstrated a mild positive correlation with varying levels of VEGF based on individual body weight and body mass index. However, Loebig et al did not evaluate the extent to which such difference in VEGF levels could impact pathophysiology of the disease, and more importantly, if such difference in VEGF levels warrant for a differential dosage of bevacizumab for treatment of AMD.

Therefore, to address this unmet need, it was postulated that due to individual differences in the VEGF levels based on body weight, a fixed dose of VEGF inhibitors, especially of Bevacizumab may not be the most optimal method for treatment of patients in all weight categories. The present invention provides the modified dosing regimen of VEGF inhibitors to cater these problems.

### SUMMARY OF THE INVENTION

In an embodiment, the invention relates to a method of treating ophthalmic diseases by administering a suitable amount of VEGF inhibitor in a patient in need thereof, wherein the suitable amount of VEGF inhibitor is determined with respect to the body weight of the said patient.

In another embodiment, the invention relates to a method of treating ophthalmic diseases by administering a suitable amount of VEGF inhibitor in a patient in need thereof, wherein a personalized dose of a suitable amount of VEGF inhibitor is administered in the intra-vitreal cavity of eye, wherein the dose to be administered is determined with respect to the body weight of the said patient at any given point in time.

In another embodiment, the invention relates to a method of treating ophthalmic diseases by administering a suitable amount of VEGF inhibitor in a patient in need thereof, wherein the suitable amount of VEGF inhibitor is determined with respect to the body weight of the said patient in order to alleviate or remove at least one of the disadvantages associated with the use of VEGF inhibitor.

In another embodiment of the invention, the VEGF inhibitor is selected from a group comprising of but not limited to Pegaptanib, Bevacizumab, Ranibizumab, Aflibercept and Brolucizumab.

In another embodiment, the invention relates to a personalized dosing for Bevacizumab for ophthalmic use, wherein the personalized dose is based on the body weight of the individual at any one point in time.

In another embodiment, the invention relates to a method of treating ophthalmic diseases by administering a suitable amount of Bevacizumab in a patient in need thereof wherein the suitable amount of Bevacizumab is determined with respect to the body weight of the said patient and the said dosage administration alleviates at least one of the adverse effects associated with the use of Bevacizumab.

In another embodiment, the invention relates to a method of treating ophthalmic diseases by administering Bevacizumab in a range of about 0.5 mg to about 0.9 mg in a patient in need thereof, wherein the patient weighs in a range from about 45 kg to about 58 Kg.

In another embodiment, the invention relates to a method of treating ophthalmic diseases by administering a suitable amount of Bevacizumab in a patient in need thereof, wherein 0.75 mg of Bevacizumab is administered to a patient, wherein the patient weighs in a range from about 45 kg to about 58 Kg.

In another embodiment, the invention relates to a method of treating ophthalmic diseases by administering Bevacizumab in a range of about 0.6 mg to about 1.2 mg in a patient in need thereof, wherein the patient weighs between 59 to 75 kg (both inclusive).

In another embodiment, the invention relates to a method of treating ophthalmic diseases by administering a suitable amount of Bevacizumab in patient in need thereof, wherein 1.0 mg of Bevacizumab is administered to a patient, wherein the patient weighs between 59-75 Kg (both inclusive).

In another embodiment, the invention relates to a method of treating ophthalmic diseases by administering Bevacizumab in a range of about 0.8 mg to about 1.5 mg in a patient in need thereof, wherein the patient weighs ≥76 Kg.

In another embodiment, the invention relates to a method of treating ophthalmic diseases by administering a suitable amount of Bevacizumab in a patient in need thereof, wherein 1.2 mg of Bevacizumab is administered to a patient, wherein the patient weighs ≥76 Kg.

In another embodiment the amount of bevacizumab administered is in the range of about 8 μg/kg to about 20 μg/kg of body weight of the patient in need thereof.

In another embodiment, the invention relates to a method of treating ophthalmic diseases by administering a suitable amount of Bevacizumab in a patient in need thereof wherein the suitable amount of Bevacizumab is selected according to body mass index.

The details of one or more embodiments of the invention set forth below are illustrative only and not intended to limit to the scope of the invention. Other features, objects and advantages of the inventions will be apparent from the description and claims.

### DESCRIPTION OF THE DRAWINGS

FIG. 1: is a flow chart of test protocol for clinical trial of weight-based dose versus fixed-doses of VEGF inhibitor in subjects with AMD.

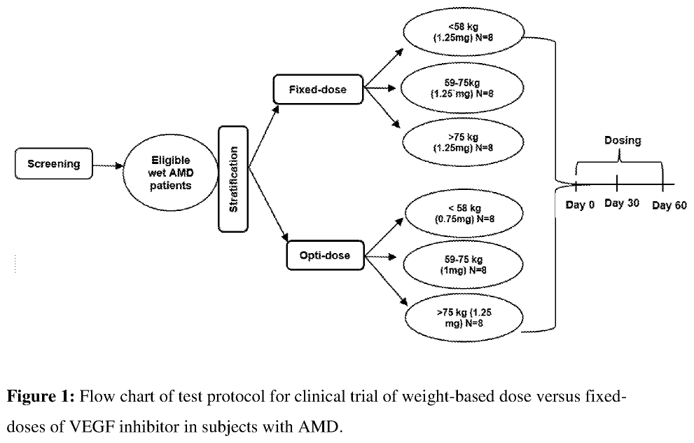

### DETAILED DESCRIPTION OF INVENTION

### Definitions

“VEGF inhibitor” means a compound which inhibits an activity or an effect of VEGF which includes but is not limited to Bevacizumab, Ranibizumab, Pegaptanib, Aflibercept, Brolucizumab, VEGF-neutralising aptamers, anti-VEGF monoclonal antibodies, siRNAs.

“About” means approximately or nearly and in the context of a numerical value or range set forth herein means±10% of the numerical value or range recited or claimed.

“Personalized dosing” means the dose amount of VEGF inhibitor is determined with respect to body weight or body mass index (BMI) of the patient.

“Ophthalmic disease” or “ocular disorder” or “ocular disease” means an eye disorder selected from age related macular degeneration (AMD) including both wet AMD and dry AMD, macular edema following retinal vein occlusion (RVO), diabetic macular edema (DME), diabetic retinopathy (DR) without or with DME, myopic choroidal neovascularization (mCNV).

“Suitable amount” or “suitable therapeutic amount” means an amount or a concentration of an active agent that has been locally delivered to an ocular region that is appropriate to safely treat an ocular condition so as to reduce or prevent a symptom of an ocular condition and essentially alleviate or reduce at least one of the disadvantage associated with the use of VEGF inhibitor. In this invention a suitable amount of VEGF inhibitor used for the treatment of ophthalmic diseases is in a range of about 0.3 mg to about 1.6 mg administered intra-vitreally in the eye.

Bevacizumab shows more side effects than Ranibizumab in most clinical trials. This is due to the systemic toxicity of Bevacizumab, and its high exposure in the vitreous. For instance, the CATT study, even a 1.25 mg dose of Bevacizumab has shown higher systemic side-effects than Ranibizumab. Table 1 represented below shows how a fixed dose of Bevacizumab would cause different systemic exposure in different patient weight group.

TABLE 1
Variable systemic exposures due to fixed dose in AMD

| | | Resulting | |
|---|---|---|---|
| | Fixed dose | per kg dose | |
| Weight | regimen | is variable | Remarks |
| 50 kg | 1.25 mg | 25 μg | Fixed dose can lead to higher |
| 75 kg | 1.25 mg | 16.6 μg | exposures (higher per kg dose) in |
| 100 kg | 1.25 mg | 12.5 μg | lower weight patients than higher |
| | | | weight patients |

It can be seen from the above Table 1 that while a 50 kg patient who receives Bevacizumab 1.25 mg would potentially be exposed to twice as much Bevacizumab.

Therefore, it is advantageous to develop a dosing regimen for treating AMD which is based on the body weight of the patient in order to minimize variability in systemic exposures thereby reducing the systemic side-effects while ensuring efficacy.

In an embodiment the invention relates to a method of treating ophthalmic diseases by administering a suitable amount of VEGF inhibitor in a patient in need thereof wherein the suitable amount of VEGF inhibitor is determined with respect to said patient body weight in order to alleviate or remove at least one of the disadvantages associated with the use of VEGF inhibitor.

In certain embodiment the invention relates to a method of treating ophthalmic diseases by administering a suitable amount of VEGF inhibitor in a patient in need thereof wherein the suitable amount of VEGF inhibitor is selected from about 0.5 mg to about 0.9 mg with respect to patient body weight and wherein the said patient body weight is in a range from about 45 kg to about 58 Kg in order to alleviate or remove at least one of the disadvantages associated with the use of VEGF inhibitor.

In another embodiment the invention relates to a method of treating VEGF associated ophthalmic diseases by administering bevacizumab in the range from about 0.5 mg to about 0.9 mg in a patient, in need thereof, wherein the patient weighs in a range from about 45 kg to about 58 Kg, and wherein the said administration alleviates or removes at least one or more of the disadvantages associated with administration of 1.25 mg of bevacizumab.

In another embodiment the bevacizumab administration in the range of 0.5 mg to about 0.9 mg in a patient weighing in a range from about 45 kg to about 58 Kg, in need thereof, demonstrates equivalent clinical effectiveness when compared to a 1.25 mg administration of bevacizumab.

In certain embodiment the invention relates to a method of treating ophthalmic diseases by administering a suitable amount of VEGF inhibitor in a patient in need thereof wherein the suitable amount of VEGF inhibitor is selected from about 0.6 mg to about 1.2 mg with respect to patient body weight and wherein the said patient body weight in the range of about 59 kg to about 75 kg (both inclusive) in order to alleviate or remove at least one of the disadvantages associated with the use of VEGF inhibitor.

In another embodiment the invention relates to a method of treating VEGF associated ophthalmic diseases by administering bevacizumab in the range from about 0.6 mg to about 1.2 mg in a patient, in need thereof, wherein the patient weighs in the range of about 59 kg to about 75 kg (both inclusive), and wherein the said administration alleviates or removes at least one or more of the disadvantages associated with administration of 1.25 mg of bevacizumab.

In yet another embodiment the bevacizumab administration in the range of 0.6 mg to about 1.2 mg to a 59 kg to 75 kg (both inclusive) patient, in need thereof, demonstrates equivalent clinical effectiveness when compared to a 1.25 mg administration of bevacizumab.

In another embodiment the invention relates to a method of treating ophthalmic diseases by administering a suitable amount of VEGF inhibitor in a patient in need thereof wherein the suitable amount of VEGF inhibitor is selected at from about 0.8 mg to about 1.5 mg with respect to patient body weight and wherein the said patient body weight ≥76 kg and above in order to alleviate or remove at least one of the disadvantages associated with the use of VEGF inhibitor.

In yet another embodiment the invention relates to a method of treating VEGF associated ophthalmic diseases by administering bevacizumab in the range from about 0.8 mg to about 1.5 mg in a patient, in need thereof, wherein the patient weighs ≥76 kg.

In yet another embodiment the bevacizumab administration in the range of 0.8 mg to about 1.5 mg in ≥76 kg patient, in need thereof, demonstrates equivalent clinical effectiveness when compared to a 1.25 mg administration of bevacizumab.

TABLE 2
Consistent systemic exposures due
to body-weight based dose in AMD:

| | | Opti Dose per Kg |
|---|---|---|
| Weight Bands (kg) | Opti Dose Broad Range | body weight of patient |
| 45-58 | 0.5-0.9 mg | 0.01-0.016 μg/Kg (8-20) |
| 59-75 | 0.6-1.2 mg | 0.01-0.016 μg/Kg (8-20) |
| ≥76 | 0.8-1.5 mg | 0.01-0.016 μg/Kg (8-20) |

In yet another embodiment the invention relates to a method of administering the VEGF inhibitor as an intravitreal injection. The needle of the syringe or the device containing the drug solution will be inserted through a pre-anesthetized conjunctiva and sclera, approximately 3.5-4.0 mm posterior to the limbus, avoiding the horizontal meridian and aiming toward the center of the globe. The angle of needle insertion through the sclera will be directed in an oblique, tunneled fashion to reduce risk of any injury. The injection volume should be delivered slowly. The needle will then be removed slowly to ensure that all drug solution is in the eye.

In yet another embodiment, the invention relates to a method of treating VEGF related ophthalmic disease, such as wet AMD by administering a suitable amount of Bevacizumab in a patient in need thereof wherein the suitable amount of Bevacizumab is selected according to body mass index.

In certain other embodiments invention provides a method to alleviate or reduce at least one of the adverse event associated with the use of Bevacizumab for treating ophthalmic diseases by administering a suitable amount of Bevacizumab in a patient in need thereof wherein the suitable amount of Bevacizumab is administered with respect to body weight of said patient.

In certain embodiments, the VEGF inhibitors are administered in patients in need by thereof by topical administration or by ocular administration. In the preferred embodiments of the present invention, VEGF inhibitors are administered by intraocular administration such as subconjunctival, intravitreal, retrobulbar, intracameral and more preferably by intravitreal administration.

The invention also provides a method to alleviate or reduce at least one of the adverse event associated with the use of Bevacizumab for treating VEGF related ophthalmic diseases by administering a suitable amount of Bevacizumab in a patient in need thereof wherein the suitable amount of Bevacizumab is selected from about 0.3 mg to about 1 mg with respect to patient body weight and preferably from about 0.5 mg to about 0.9 mg with respect to patient body weight, wherein the body weight of said patient is in a range from about 45 kg to about 58 Kg.

The invention also provides a method to alleviate or reduce at least one of the adverse event associated with the use of Bevacizumab for treating ophthalmic diseases by administering a suitable amount of Bevacizumab in a patient in need thereof wherein the suitable amount of Bevacizumab is selected from about 0.4 mg to about 1.2 mg with respect to patient body weight and preferably from about 0.6 mg to about 1.2 mg with respect to patient body weight, wherein the body weight of said patient is in the range of about 59 kg to about 75 kg (both inclusive).

The invention also provides a method to alleviate or reduce at least one of the adverse event associated with the use of Bevacizumab for treating ophthalmic diseases by administering a suitable amount of Bevacizumab in a patient in need thereof wherein the suitable amount of Bevacizumab is selected from about 0.5 mg to about 1.6 mg with respect to patient body weight and preferably from about 0.8 mg to about 1.5 mg with respect to patient body weight, wherein the said patient body weight is ≥76 kg.

The invention also provides a method to alleviate or reduce at least one of the adverse event associated with the use of Bevacizumab for treating ophthalmic diseases by administering a suitable amount of Bevacizumab in a patient in need thereof wherein the suitable amount of Bevacizumab is selected either from (1) about 0.3 mg to about 1 mg with respect to patient body weight and preferably from about 0.5 mg to about 0.9 mg with respect to patient body weight, wherein the body weight of said patient is in a range from about 45 kg to about 58 Kg or (2) about 0.4 mg to about 1.2 mg with respect to patient body weight and preferably from about 0.6 mg to about 1.2 mg with respect to patient body weight, wherein the body weight of said patient in the range of about 59 kg to about 75 kg (both inclusive) or (3) about 0.5 mg to about 1.6 mg with respect to patient body weight and preferably from about 0.8 mg to about 1.5 mg with respect to patient body weight, wherein the said patient body weight is ≥76 kg, wherein the adverse event associated with intra-vitreal use of Bevacizumab leads to two types of adverse effects; ocular (local side effect) and non-ocular (systemic side-effects due to leaking of Bevacizumab into circulation). The local side-effects include vitreous detachment, inflammation, hemorrhage and infection of eye. The most common systemic adverse events include hypertension, gastrointestinal symptoms (hemorrhage, nausea and vomiting), and upper respiratory infections (pneumonia) etc.

In yet another embodiment, the invention provides the reduction of about 40% in the amount of bevacizumab to be administered to the patient with respect to the body weight in comparison to the standard amount of 1.25 mg, wherein the patient has a body weight in a range from about 45 kg to about 58 Kg.

In yet another embodiment, the invention provides the reduction of about 20% in the amount of bevacizumab to be administered to the patient with respect to the body weight in comparison to the standard amount of 1.25 mg, wherein the patient has body weight in a range of 59-75 kg (both inclusive).

In yet another embodiment, the invention relates to a method of treating ophthalmic diseases by administering a suitable amount of Ranibizumab in a patient in need thereof wherein the suitable amount of Ranibizumab is selected according to body mass index.

In yet another embodiment, the invention relates to a method of treating ophthalmic diseases by administering a suitable amount of Aflibercept in a patient in need thereof wherein the suitable amount of Aflibercept is selected according to body mass index.

In yet another embodiment, the invention relates to a method of treating ophthalmic diseases by administering a suitable amount of Brolucizumab in a patient in need thereof wherein the suitable amount of Brolucizumab is selected according to body mass index.

In one more embodiment, the present invention provides comparative study of the fixed-dosage versus weight-adjusted dosage of VEGF inhibitors that can be presented based on following study endpoints:

Comparison of systemic Peak Concentration (Cmax) of Fixed-dose versus Opti-dose
Comparison of systemic Area Under the Curve (AUC) of Fixed-dose versus Opti-dose
Comparison of systemic VEGF concentration of Fixed-dose versus Opti-dose
Comparison of proportion of patients losing fewer than 15 letters (approximately 3 lines) from baseline visual acuity at end of three months between the groups.
Comparison of mean change in best corrected visual acuity (BCVA) from baseline at the end of three months between the groups
Comparison of side-effects profile between the groups.

### Example 1— POC Clinical Trial of Weight-Based Dose Versus Fixed-Doses of Bevacizumab in Subjects with Age-Related Macular Degeneration

A multi-center, open-label, non-randomized, comparative, exploratory, proof-of-concept clinical study in wet-AMD patients was undertaken to compare Pharmacokinetics (PK), Pharmacodynamics (PD), Safety & Efficacy of intravitreal fixed-dose (1.25 mg) versus weight-adjusted dosing (Opti-dose) of Bevacizumab.

In this study, 48 patients of either gender ≥50 years of age with diagnosis of active subfoveal CNV lesions secondary to age-related macular degeneration were planned to be enrolled from two sites according to weight bands (<58 kg, 59-75 kg, ≥75 kg). Eligible patients were allocated to one of the three treatment arms based on body weight to receive Bevacizumab 0.75 mg, 1 mg or 1.25 mg, intravitreally. Each patient received one injection every month for three months (total 3 injections) at Day 1, Day 30 and Day 60 and were followed-up for 1 month following last injection.

Visual acuity was assessed with ETDRS chart (Early Treatment Diabetic Retinopathy Study Chart) at 4 meter distance. Serum Bevacizumab levels and systemic VEGF levels were to be assessed at pre-dose (Day 1), 3 hours post dose (Day 1) and on Day 3 (72 hours), Day 7, Day 15, Day 30, Day 60 (pre-dose and 3 hours post-dose), Day 67, and Day 90. Day 90 was End of the Study (EOS) visit. The efficacy was evaluated in terms of validated clinical endpoints i.e. mean change in BCVA and proportion of patients losing fewer than 15 letters (approximately 3 lines) from baseline at the end of three months (Day 90). Incidence of drug related adverse events as assessed by clinical and ophthalmic examinations, vital and laboratory parameters, ECG.

Review of data from first 23 patients who completed the study, exhibited promising results clearly indicating weight adjusted doses providing similar efficacy benefits compared to fixed doses.

The Efficacy results from these 23 completed patients is provided below:

TABLE 3
Mean Change in BCVA and Responder Rate for Opti-dose Vs Fixed dose of Bevacizumab

| | Patients Body Weight (limits inclusive) in Kg | | | | | | | | | |
|---|---|---|---|---|---|---|---|---|---|---|
| | 45-58 | | | | 59-75 | | | | >75 | |
| Dosage | 0.75 mg | (0.03 mL) | 1.25 mg | (0.05 mL) | 1 mg | (0.04 mL) | 1.25 mg | (0.05 mL) | 1.25 mg | (0.05 mL) |
| N | 4 | | 4 | | 6 | | 5 | | 4 | |
| Mean change of BCVA | 12 | (12.9) | 1 | (12.4) | 16 | (13.9) | 6.8 | (3.8) | 5.25 | (7.3) |
| from baseline to week | | | | | | | | | | |
| [Mean (SD)] | | | | | | | | | | |
| Comparison of change | 0.41 | | | | 0.25 | | | | NA | |
| in BCVA between doses | | | | | | | | | | |
| (p value) | | | | | | | | | | |
| Responder Rate (%) | 100% | (3/3) | 75% | (3/4) | 100% | (4/4) | 100% | (5/5) | 100% | (4/4) |
| amongst completers | | | | | | | | | | |

| | | | |
|---|---|---|---|
| Comparison of | 0.34 | 0.45 | NA |
| responder rate | | | |
| (p value) | | | |

From above results, it is evident that there is no significant difference observed in mean change in BCVA score from baseline between lower doses as per weight band as compared to the fixed higher dose in the same weight group. Further, there is no significant difference observed in the responder rates between lower doses administered as per weight band as compared to fixed higher dose in the same weight group. In conclusion comparable mean BCVA change and responder rate in terms of BCVA gain are observed in Bevacizumab when administered as optimal lower dose (0.75 mg and 1 mg) as per weight bands as compared to fixed higher dose (1.25 mg). Three AEs occurred in three patients (one in each dose group) all of which were either not related or unlikely related to study drug inferring comparable safety between the opti-dose and higher dose groups anticipating that systemic VEGF levels for 0.75 and 1 mg would be higher as compared to 1.25 mg in lower weight groups thus acting as a surrogate marker of better safety profile in terms of serious side effects, VEGF being constitutive in nature.

All patents, patent applications and publications cited in this application are hereby incorporated by reference in their entirety for all purposes to the same extent as if each individual patent, patent application or publication were so individually denoted.

Although certain embodiments and examples have been described in detail, persons having ordinary skill in the art will clearly understand that many modifications are possible in the embodiments and examples without departing from the teachings thereof.

1. A method of treating an ocular disease in a patient comprising administering to the patient a weight-adjusted dose of a VEGF inhibitor, wherein the weight-adjusted dose is selected from the group comprising of
about 0.5 mg to about 0.9 mg of VEGF inhibitor in the patient body weighing in the range from about 45 kg to about 58 Kg;
about 0.6 mg to about 1.2 mg of VEGF inhibitor in the patient body weighing in the range from about 59 kg to about 75 Kg; and
about 0.8 mg to about 1.5 mg of VEGF inhibitor in the patient body weighing more than 75 Kg.

2. The method according to claim 1, wherein the VEGF inhibitor is selected from Pegaptanib, Bevacizumab, Ranibizumab, Aflibercept and Brolucizumab.

3. The method according to claim 1, wherein the weight-adjusted dose of VEGF inhibitor administered is 0.75 mg in the patient body weighing between 45 to 58 Kg.

4. The method according to claim 1, wherein the weight-adjusted dose of VEGF inhibitor administered is 1.0 mg in the patient body weighing between 59 to 75 Kg.

5. The method according to claim 1, wherein the weight-adjusted dose of VEGF inhibitor administered is 1.2 mg in the patient body weighing more than 75 Kg.

6. The method according to claim 1, wherein the VEGF inhibitor is administered in the range of about 8 μg/kg to about 20 μg/kg of body weight of the patient.

7. The method according to claim 1, wherein the ocular disease is selected from neovascular (wet) age-related macular degenerations, dry macular degenerations, diabetic macular edema, diabetic retinopathy, central retinal vein occlusion, corneal neovascularization or branched retinal vein occlusion.

8. The method according to claim 1, wherein ocular disease is neovascular (wet) age-related macular degeneration.

9. The method according to claim 1, wherein ocular disease is diabetic macular edema.

10. The method according to claim 1, wherein VEGF inhibitor is administered to the patient by intraocular administration.

11. The method according to claim 1, wherein VEGF inhibitor is administered to the patient by intravitreal administration.

12. A method of treating an ocular disease in a patient comprising administering to the patient a weight-adjusted dose of a Bevacizumab, wherein the weight-adjusted dose is selected from the group comprising of
about 0.5 mg to about 0.9 mg of Bevacizumab in the patient body weighing in the range from about 45 kg to about 58 Kg;
about 0.6 mg to about 1.2 mg of Bevacizumab in the patient body weighing in the range from about 59 kg to about 75 Kg; and
about 0.8 mg to about 1.5 mg of Bevacizumab in the patient body weighing more than 75 Kg.

13. The method according to claim 12, wherein the weight-adjusted dose of Bevacizumab administered is 0.75 mg in the patient body weighing between 45 to 58 Kg.

14. The method according to claim 12, wherein the weight-adjusted dose of Bevacizumab administered is 1.0 mg in the patient body weighing between 59 to 75 Kg.

15. The method according to claim 12, wherein the weight-adjusted dose of Bevacizumab administered is 1.2 mg in the patient body weighing more than 75 Kg.

16. The method according to claim 12, wherein the Bevacizumab is administered in the range of about 8 μg/kg to about 20 μg/kg of body weight of the patient.

17. The method according to claim 12, wherein the ocular disease is selected from neovascular (wet) age-related macular degenerations, dry macular degenerations, diabetic macular edema, diabetic retinopathy, central retinal vein occlusion, corneal neovascularization or branched retinal vein occlusion.

18. The method according to claim 12, wherein ocular disease is neovascular (wet) age-related macular degeneration.

19. The method according to claim 12, wherein ocular disease is diabetic macular edema.

20. The method according to claim 12, wherein Bevacizumab is administered to the patient by intraocular administration.

21. The method according to claim 12, wherein Bevacizumab is administered to the patient by intravitreal administration.
